(19) United States
(12) Patent Application Publication
(10) Pub. No.: US 2010/0318167 A1
(43) Pub. Date: Dec. 16, 2010

(54) NEUROSTIMULATION ELECTRODE ARRAY AND METHOD OF MANUFACTURE

(75) Inventors: Brian M. Conn, Broomfield, CO (US); James R. Easter, Lyons, CO (US); Farid Moumane, Lafayette, CO (US); Michel Bruhwiler, Newton, MA (US); Ira Spool, Brookline, MA (US); Melissa Rosen, Salem, MA (US)

Correspondence Address: MARSH, FISCHMANN & BREYFOGLE LLP, 8055 East Tufts Avenue, Suite 450, Denver, CO, 80237, US

(73) Assignee: Otologics, LLC, Boulder, CO (US)

(21) Appl. No.: 12/762,540

(22) Filed: Apr. 19, 2010

Related U.S. Application Data
(60) Provisional application No. 61/170,389, filed on Apr. 17, 2009.

(51) Int. Cl. A61N 1/05

(52) U.S. Cl. 607/148

(57) ABSTRACT

An implantable neurostimulation electrode array may include a flexible, electrically non-conductive carrier, and a plurality of electrodes supportably interconnected to and spaced along a length of a carrier. In various embodiments, different ones of at least some of the plurality of electrodes may comprise corresponding contact surfaces having different areas. The different surface areas may be achieved by defining a different area per unit length of the carrier and/or by defining a different total length (e.g. as measured along a length of the carrier). In some embodiments, insulator portions having different corresponding lengths may be disposed between different adjacent ones of the electrodes. The insulated portions may be integrally defined with the carrier. Electrical connection lines may be embedded within the carrier. In a manufacturing method, a plurality of electrodes may be supportably interconnected to a first side of a sacrificial substrate and a carrier may be interconnected to the electrodes. In turn, at least a portion of the substrate may be removed from the electrodes to yield at least a partially completed electrode array. The substrate may be configured into a predetermined configuration wherein the carrier material may be formed within an internal volume thereof to define at least a portion of the configuration of the carrier of the electrode array.

### RELATED APPLICATIONS

This application claims priority to U.S. Provisional Application Ser. No. 61/170,389, filed Apr. 17, 2009, entitled “NEUROSTIMULATION ELECTRODE AND METHOD OF MANUFACTURE”, the entirety of which is hereby incorporated by reference.

### FIELD OF THE INVENTION

The present invention relates to neurostimulation, and more particularly to an implantable neurostimulation electrode array and manufacturing method that facilitates enhanced production capabilities and product customization/performance.

### BACKGROUND OF THE INVENTION

The use of neurostimulation implant devices is ever-increasing. Such devices employ a plurality of implanted electrodes that are selectively activated to affect a desired neuro-response, including sound sensation, pain/tremor management, and urinary/anal incontinence. By way of primary interest, auditory neurostimulation implant devices include auditory brainstem implant (ABI) and cochlear implant (CI) devices.

In the case of CI devices, an electrode array is inserted into the cochlea of a patient, e.g. typically into the scala tympani so as to access and follow the spiral curvature of the cochlea. The array electrodes are selectively driven to stimulate the patient's auditory nerve endings to generate sound sensation. In this regard, a CI electrode array works by utilizing the tonotopic organization, or frequency-to-location mapping, of the basilar membrane of the inner ear.

In a normal ear, sound vibrations in the air are transduced to physical vibrations at the tympanic membrane and communicated by the ossicular chain to the oval window, and in turn, to the basilar membrane inside the cochlea. High frequency sounds do not travel very far along the membrane, while lower frequency sounds pass further along. The movement of hair cells, located along the basilar membrane, creates an electrical disturbance, or potential, that can be picked up by auditory nerve endings that generate electrical action pulses that travel along the auditory nerve to the brainstem. In turn, the brain is able to interpret the nerve activity to determine which area of the basilar membrane is resonating, and therefore what sound frequency is being sensed. By directing which electrodes of a CI electrode array are activated, cochlear implants can selectively stimulate different parts of the cochlea and thereby convey different acoustic frequencies corresponding with a given audio input signal.

With ABI systems a plurality of electrodes may be implanted at a location that bypasses the cochlea. More particularly, an array of electrodes may be implanted at the cochlea nucleus, or auditory cortex, at the base of the brain to directly stimulate the brainstem of a patient. Again, the electrode array may be driven in relation to the tonotopic organization of a recipient's auditory cortex to obtain the desired sound sensation.

As may be appreciated, in the case of either ABI electrodes or CI electrodes, audio signals (e.g. from a microphone) may be processed, typically utilizing what is referred to as a speech processor, to generate stimulation signals utilized to selectively drive the electrodes for stimulated sound sensation. Further, in both implant approaches a source of power may be included to power the stimulation signal generator.

### SUMMARY OF THE INVENTION

One objective of the present invention is to provide an improved neurostimulation electrode array and/or method of manufacture thereof that facilitates the realization of improved production efficiencies and repeatability.

Another objective of the present invention is to provide an improved neurostimulation electrode array and/or method of manufacture thereof that facilitates product customization.

Yet another objective of the present invention is to provide an improved neurostimulation electrode array and/or method of manufacture thereto that yields enhanced product reliability and/or performance.

In relation to one or more of the noted objectives, the present inventors have recognized the desirability of an implantable neurostimulation electrode array having at least some electrodes of differing configurations, e.g. differing surface areas and/or differing surface shapes and/or differing surface distances from a longitudinal axis of the array. The present inventors have also recognized the desirability of an implantable neurostimulation electrode array having electrically non-conductive array portions between electrodes, wherein at least some of such non-conductive portions have differing configurations, e.g. differing surface lengths along and/or differing surface distances from a longitudinal axis of the array. The present inventors have further recognized the desirability of an implantable neurostimulation electrode array having one or more electrodes whose surface is of a non-uniform or complex shape along and/or about a longitudinal axis of the array.

In relation to one or more of the above-noted objectives, the present inventors have also recognized the desirability of an implantable neurostimulation electrode array manufacturing approach that utilizes a sacrificial substrate to facilitate electrode array assembly. In conjunction therewith, the present inventors have recognized the desirability of utilizing such a manufacturing approach to provide an implantable neurostimulation electrode array having one or more of the above-noted configuration attributes.

In relation to the foregoing, an implantable neurostimulation electrode array may be provided that includes a flexible, electrically non-conductive carrier. Additionally, the electrode array may include a plurality of electrodes supportably interconnected to and spaced along a length of a carrier.

In one aspect, the electrode array may be provided so that different ones of at least some of the plurality of electrodes comprise corresponding different contact surfaces having different areas. The different contact surfaces may be provided to have at least one of a different area per unit length of the carrier and/or a different length as measured along a length of the carrier. In one approach, the different contact surfaces may comprise at least corresponding portions that are disposed at different distances from a longitudinal axis of the carrier.

In a related aspect, each of the different contact surfaces may extend about and along a longitudinal axis of the carrier. In this regard, each of the different contact surfaces may be of an annular configuration. More particularly, in one implementation each of the plurality of electrodes may be of a ring-shaped configuration with the carrier being located to extend therethrough. By way of example, at least some of the ring-shaped electrodes may be located at different radial distances from a longitudinal center axis.

In yet another aspect, different ones of at least some of the plurality of electrodes of the implantable neurostimulation electrode array may be spaced at different distances from adjacent ones of said plurality of electrodes, e.g. as measured along a length of the carrier. In this regard, spaces between adjacent ones of the plurality of electrodes may comprise electrically non-conductive, insulator portions of the electrode array, wherein at least some of the insulator portions are of different configurations, e.g. different lengths.

In a related aspect, insulator portions of an electrode array may be provided so that contact surfaces of at least some of the plurality of electrodes may be recessed relative to one or both of the adjacent insulator portions. In one implementation, insulator portions of the array may be provided to define an outer periphery having a square wave, or square tooth, configuration along a length of the array. In turn, contact surfaces of the plurality of electrodes may be supportably disposed on recessed surfaces of the array located between adjacent raised-surfaces of the array that define each square-tooth thereof. In one embodiment, raised-surfaces of the array may be provided to define a tapered-down array configuration from a proximal end to a distal end thereof. In another embodiment, insulator portions may be provided to define recessed surfaces therebetween of differing depths, wherein contact surfaces of at least some of the electrodes are located within corresponding recesses at differing recessed distances from an outer periphery of the array.

In a further aspect, an implantable neurostimulation electrode array may be provided with insulator portions disposed between adjacent ones of said plurality of electrodes, wherein the insulator portions are integrally defined by the carrier of the electrode array. In this regard, the insulator portions and carrier may be defined in a single operation.

In a related aspect, the electrode array may further comprise a plurality of electrical signal lines supportably interconnected to the carrier, wherein at least some of the electrical signal lines are electrically interconnected to different ones of the plurality of electrodes, and wherein each of the plurality of electrical signal lines extend through at least a portion of the carrier. In the later regard, the electrical signal lines may be sealably disposed within the carrier. More particularly, in one implementation the electrical signal lines may be embedded within the carrier, e.g. contemporaneous with the formation of the carrier and insulator portions located between adjacent pairs of the electrodes.

In yet an additional aspect, one or more electrodes of a neurostimulation electrode array may be provided to have a contact surface with a shape that varies as it extends about or along a longitudinal axis of the array. For example, a width of a given electrode may increase and/or decrease as it progresses around a carrier, e.g. to define a lens-like shape, a bowtie-like shape, etc. In a related aspect, the electrode array may be provided so that different ones of at least some of the plurality of electrodes comprise corresponding different contact surfaces having different complex or non-uniform shapes.

An inventive method for manufacture of an implantable neurostimulation electrode array is also provided. The method may include the steps of providing a plurality of electrodes supportably connected to a first side of a substrate, and interconnecting a carrier to the plurality of electrodes. In turn, the method may further include the step of removing at least a portion of the substrate from each of the plurality of electrodes after the carrier interconnection step. As may be appreciated, the utilization of a substrate that is at least partially sacrificed during the production process may yield numerous production advantages as well as enhanced electrode array reliability, performance and customization capabilities.

In one aspect, the method may further include the step of configuring the substrate into a predetermined configuration so that the first side of the substrate and exposed first surfaces of the plurality of electrodes face inward, and so that an opposing second side of the substrate and covered second surfaces of the plurality of electrodes (e.g. opposing the exposed first surfaces) face outward, wherein the carrier interconnection step is completed with the substrate in the predetermined configuration. In the later regard, the interconnecting step may be completed by forming a bio-compatible, electrically non-conductive carrier material within an internal volume defined by the predetermined configuration of the substrate. In certain embodiments, the internal volume may advantageously define a peripheral configuration corresponding with a desired configuration of at least a portion of the electrode array. As may be appreciated, the carrier material may bond to the exposed first surfaces during carrier formation. In turn, upon substrate removal, the covered second surfaces of the electrodes may be at least partially exposed to define electrode contact surfaces for electrical signal delivery to/receipt from contacted tissue.

In one approach, the interconnecting step may include changing the state of the carrier material, wherein the carrier material bonds to exposed surfaces of the plurality of electrodes. By way of example, a change-of-state may include temperature elevation of the carrier material so as to flow the carrier material and contemporaneously define an integral carrier structure. In one implementation, the substrate may be configured into a predetermined configuration sized for positioning into a complimentary mold, wherein the interconnecting step may include injection molding of a carrier material into an internal volume defined by the predetermined configuration of the substrate.

In another aspect, the provision of the plurality of electrodes in the method may be completed so that different ones of at least some of the plurality of electrodes comprise corresponding different contact surfaces having different areas. In this regard, the different contact surfaces may be provided to have at least one of a different area per unit length of the carrier and/or a different length as measured along the length of the carrier.

In one approach, the provision of electrodes may comprise the steps of disposing a metal layer on the substrate, and removing predetermined portions of the metal layer to define the plurality of electrodes. In the later regard, the removing step may be completed so as to realize the above-noted feature of providing different electrodes with contact surfaces having different surface areas. Alternatively or additionally, the removing step may be completed so as to define spaces of different predetermined configurations between different adjacent pairs of the electrodes, e.g. spaces having different lengths between different ones of the electrodes as measured along a longitudinal axis.

As may be appreciated, the disposing of a metal layer may include metalizing the layer by any one of a number of different techniques, including for example, plating, sputtering, electrodeposition, vapor deposition, and electroless plating. Further, the removal of portions of the metal layer step may encompass any of a number of techniques, including etching selected portions of the metal layer by milling, plasma-based, sputter or ion beam etching, electrochemical machining or hydromachining.

In another approach, the provision of electrodes may include the step of disposing metal at a plurality of discrete, spaced locations to define the plurality of electrodes. In this regard, prior to such disposing step the method may further comprise a step of removing portions of the first side of the substrate to define elevated regions, wherein different ones of the plurality of spaced locations for metal disposition are located on different ones of the elevated regions. In conjunction with this step, the removed substrate portions may be of different configuration, e.g. of different lengths as measured along a longitudinal axis so as to yield different predetermined spacing between different adjacent pairs of the electrodes. In yet a further aspect, the inventive method may include the step of connecting different ones of a plurality of electrical connection lines to exposed surfaces of different ones of the plurality of electrodes prior to the first, above-noted carrier interconnection step. In this regard, the plurality of electrical connection lines may be embedded within the carrier. In one implementation, such embedding may occur in conjunction with the step of interconnecting the carrier to a plurality of electrodes.

In yet a further aspect, the provision of a plurality of electrodes on a substrate may be completed with the substrate disposed in a substantially planar orientation. Further, such substantially planar orientation may be maintained during a further step of connecting electrical connection lines to exposed surfaces of different ones of the plurality of electrodes.

In an additional aspect, the substrate removal step may be completed via any number of a plurality of techniques that may provide for selectively changing the state of the substrate without adversely affecting the electrodes, carrier or interconnected electrical connection lines. In one approach, substrate removal may be achieved by dissolving at least a portion of the substrate utilizing an acidic solution. In other approaches, plasma-based, sputter or ion beam etching may be used for substrate removal.

As may be appreciated, various aspects of the noted methodology may be utilized to yield a neurostimulation electrode array having various ones or all of the above-noted electrode array features. Numerous additional advantages and aspects of the present invention will become apparent to those skilled in the art upon review of the further description that follows.

### BRIEF DESCRIPTION OF THE DRAWINGS

FIG. 1 is a perspective view of one embodiment of a neurostimulation electrode array.

FIG. 2 is a cross-sectional side view of the neurostimulation electrode array embodiment of FIG. 1.

FIG. 3 is a top view of a portion of an assembly comprising a plurality of electrodes, a substrate and interconnected electrical connection lines, employable in one embodiment of a method for manufacture of a neurostimulation electrode array.

FIG. 4 is a side view of the assembly portion shown in FIG. 3.

FIG. 5 is perspective view of the assembly portion shown in FIGS. 3 and 4, wherein the assembly portion has been configured from the open planar configuration shown in FIGS. 3 and 4 to a frusto-conical tubular configuration.

### DETAILED DESCRIPTION OF THE DRAWINGS

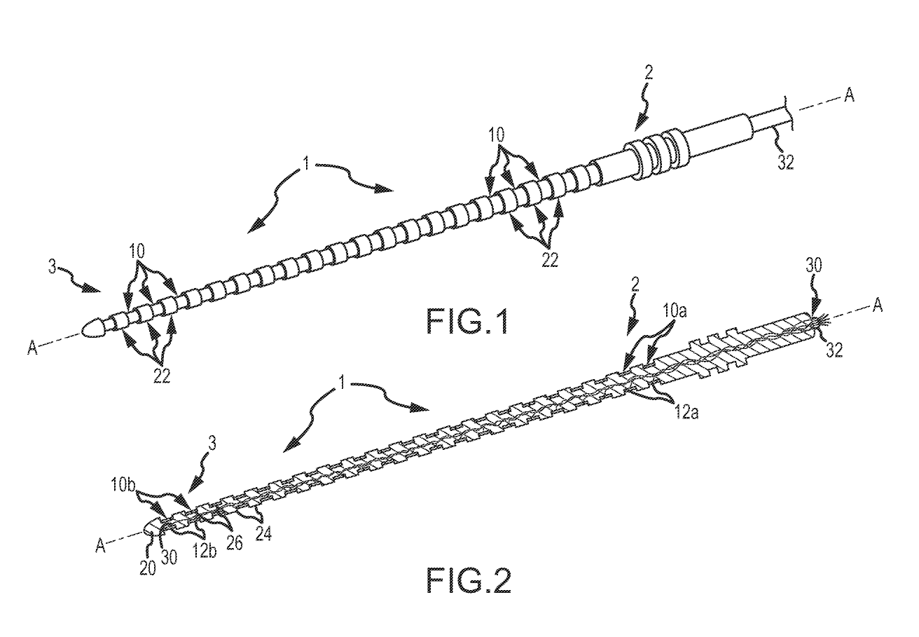

FIGS. 1 and 2 illustrate one embodiment of a neurostimulation electrode array 1 adapted for cochlear implant use. In such an implementation, the electrode array 1 may be inserted into a cochlea of a patient, wherein electrical stimulation signals may be applied to various ones of a plurality of electrodes to yield nerve stimulation for auditory perception. As may be appreciated, the features of the neurostimulation electrode array 1 are employable in other embodiments of the present invention.

In the illustrated embodiment, the neurostimulation electrode array 1 may be of an elongated configuration having a plurality of electrically-conductive electrodes 10 positioned about and spaced along a longitudinal axis AA. The electrodes 10 may include corresponding contact surfaces 12 for contacting tissue and transmitting and/or receiving signals when implanted.

As shown in FIG. 2, the electrodes 10 may be supportably interconnected to a carrier 20 that extends through the electrodes 10 from a proximal end 2 to a distal end 3 of the neurostimulation electrode array 1. The carrier 20 may include electrically non-conductive insulator portions 22 disposed between different pairs of the electrodes 10. In one approach, the carrier 20 may integrally define the insulator portions 22. In other approaches, the carrier may include one or more internal layers of material extending along the length thereof with insulator portions 22 separately defined and interconnected to an outside surface thereof.

The outer surface of the carrier 20 may be of a square wave, or square-toothed configuration, wherein alternating raised surfaces 24 and recessed surfaces 26 are provided. In the embodiment shown in FIG. 2, the raised surfaces 24 may be provided on the insulator portions 22 and the electrodes 10 may be supportably disposed on the recessed surfaces 26. As further shown, the raised surfaces 24 may project outward beyond the contact surfaces 12 of adjacent electrodes 10. In this regard, the raised surfaces 24 serve to focus and direct the transfer of charge. In a cochlear implant, this property may be used to enhance frequency resolution.

The carrier 20 may also be provided so that raised surfaces 24 and recessed surfaces 26 have annular, or ring-shaped, cylindrical configurations having a common center axis corresponding with longitudinal axis AA. As a further option, the raised surfaces 24 may be provided such that at least a portion of one or more raised surface(s) 24a located near the proximal end 2 is disposed at an offset distance from the longitudinal axis AA that is greater than any portion of one or more raised surface(s) 24b located near the distal end 3. Similarly, at least a portion of one or more recessed surface(s) 26a located near the proximal end 2 may be disposed at an offset distance from the longitudinal axis AA that is greater than any portion of one or more recessed surface(s) 26b located near the distal end 3.

In the illustrated arrangement, at least some of the raised surfaces 24 and/or at least some of recessed surfaces 26 may be disposed at corresponding, decreasing distances from the longitudinal axis AA from the proximal end 2 to the distal end 3. Similarly, at least some of the contact surfaces 12 of electrodes 10 may be disposed at corresponding, decreasing distances from the longitudinal axis AA from the proximal end 2 to the distal end 3. As best shown in FIG. 2, the raised surfaces 24 of the illustrated embodiment define a tapered down configuration from the proximal end 2 to the distal end 3.

Of note, at least some of the electrodes 10 may be provided to have corresponding contact surfaces 12 with different contact surface areas. In one approach, at least some of the contact surfaces 12 may be provided to have different areas per unit length as measured along the longitudinal axis AA. For example, the illustrated embodiment of FIGS. 1 and 2, electrodes 10 may have decreasing contact surface areas (e.g. due to decreasing circumferences) as a function of their location from the proximal end 2 to the distal end 3. That is, contact surface(s) 12a provided on electrode(s) 10a near the proximal end 2 may be provided to have a greater contact surface area(s) than the contact surface area(s) of a contact surface(s) 12b of electrode 10b located near the distal end 3.

In other embodiments, the distance of contact surfaces 12 of electrodes 10 from longitudinal axis AA (e.g. the radial location thereof) may be selectively established along the length of the neurostimulation electrode 1 so as to increase from the proximal end 2 to the distal end 3, or so as to increase, decrease and/or be equal from electrode-to-electrode.

Alternatively or additionally, and while not shown in FIGS. 1 and 2, the surface areas of contact surfaces 12 of electrodes 10 may be also selectively established to be different along the length of the neurostimulation electrode array 1 by simply varying the length of the electrodes 10 as measured along the longitudinal axis AA. For example, the length of contact surfaces 12 of electrodes 10 may increase, decrease and/or be equal from electrode-to-electrode. Further, one or more of the contact surfaces 12 of electrodes 10 may be provided to have a non-uniform or complex configuration.

Of further note, the insulator portions 22 of neurostimulation electrode array 1 may be provided so that different ones thereof have different corresponding lengths and/or other outer configuration differences. By way of example, the length of selected ones of the insulator portions 22 may be established to selectively locate electrodes 10 with different spacing between different pairs thereof (e.g. different spacing lengths). In this regard, insulator portions 22 may be selectively varied with or without selectively varying the surface area(s) of contact surfaces 12 of electrodes 10. Further, the insulator portions 22 may be provided to locate contact surfaces 12 of electrodes 10 at differing recessed distances relative to the outer periphery of the array 1.

As shown in FIG. 2, the neurostimulation electrode array 1 may further comprise a plurality of electrical connection lines 30. Different ones of the electrical connection lines 30 may be electrically interconnected to different ones of the plurality of electrodes 10 to facilitate the selected provision of different electrical stimulation signals thereto and/or receipt of signals therefrom. As shown, the electrical interconnection lines 30 may extend into the carrier from the proximal end thereof and extend through the carrier 20 to the location of a corresponding interconnected electrode 10. Further in this regard, the electrical connection lines 30 may be sealably disposed within the carrier 20. In various arrangements, the electrical connection lines 30 may be embedded within the carrier 20.

In the illustrated arrangement, the electrical connection lines 30 may comprise corresponding electrical wires bundled together and sealably enclosed within a cable line 32 extending away from the proximal end 2 of the neurostimulation electrode array 1. As may be appreciated, the cable line 32 may electrically interface with other implanted componentry for electrical signal transmission between such componentry and the neurostimulation electrode array 1.

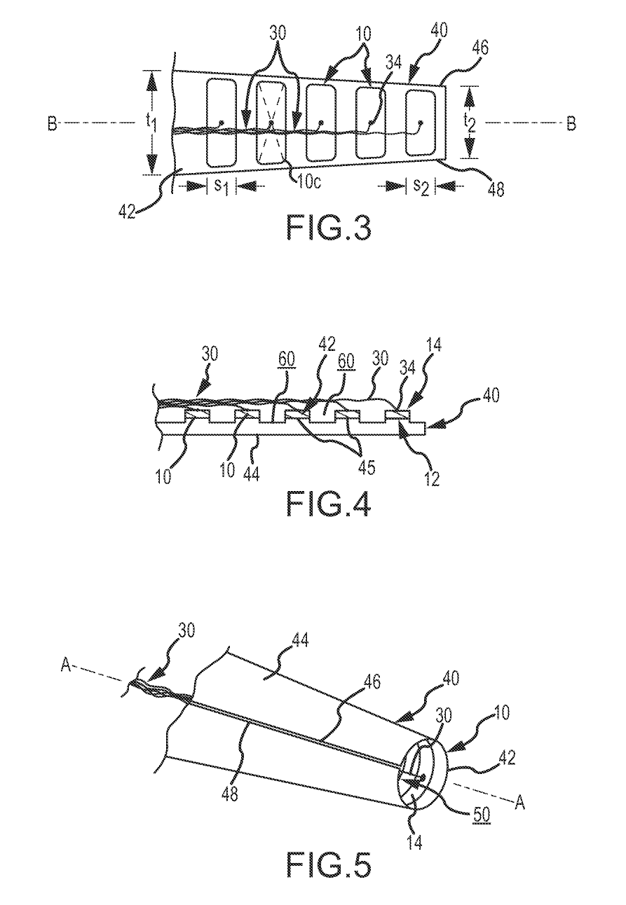

Reference is now made to FIGS. 3-5, which illustrate one embodiment of a method for manufacture of an implantable neurostimulation electrode array. By way of example, such method embodiment may be employed for manufacture of the embodiment of neurostimulation electrode array 1 of FIGS. 1 and 2, and the description that follows will relate thereto. As may be appreciated, the method embodiment may be employed in conjunction with the manufacture of various other array electrode embodiments as well.

In general relation to the described method embodiment, electrodes may be supportably connected to a sacrificial substrate and to a carrier. Then, at least a portion of the substrate may be removed to yield at least a partially completed electrode.

More particularly, FIGS. 3-5 illustrate a portion of an in-process assembly, wherein a plurality of electrodes 10 may be supportably connected to a substrate 40 on a first side 42 thereof. As shown, the electrodes 10 may be provided to define open spaces, or recesses, 60 therebetween. Further, the electrodes 10 may be disposed on elevated regions 45 of the substrate 40. The electrodes 10 may be formed on substrate 40 in a number of different approaches.

In one approach, a metal layer may be disposed on the substrate 40 and portions of the metal layer may be selectively removed to define the electrodes 10 and open spaces 60 therebetween. In this regard, the location, amount and/or configuration of the removed portions of substrate 40 may be selectively established to yield different configurations and sizes of electrodes 10 and/or to yield different configurations and sizes of the spaces 60 therebetween. As will be further described hereinbelow, the spaces 60 may be filled with a carrier material define corresponding insulator portions 22 of a carrier 20, as shown in FIGS. 1 and 2 above.

By way of example, the metal layer may be provided by a metallization process, e.g. via plating, sputtering, electrodeposition, vapor deposition, and electroless plating. Selective removal of portions of the metal layer may also be achieved by a number of different techniques, including etching by milling, plasma-based, sputter or ion beam etching, electrochemical machining or hydromachining.

In conjunction with removal of selected portions of a metal layer, underlying portions of substrate 40 may also be removed to define pockets (e.g. so as to increase the depth of spaces 60) with elevated substrate regions 45 therebetween.

Such electrode regions 45 correspond with the location of electrodes 10. In turn, and as will become apparent below, the pockets and corresponding spaces 60 may be filled with a carrier material to yield insulator portions 22 of a carrier 20 that project outwardly beyond the contact surfaces 12 of adjacent electrodes 10 in an electrode array 1. By varying the height at the electrode regions 45 (e.g. which correspondingly varies the depth of the pockets), the contact surfaces 12 of electrodes 10 may be disposed at different recessed distances from an outer periphery of the electrode array 1. In other approaches, electrodes 10 may be formed by selectively and separately applying metal to only the corresponding locations on substrate 40 where electrodes 10 are desired. By way of example, in one implementation metal may be plated at predetermined separate locations to define electrodes 10. In another implementation metal pads may be preformed and connect to substrate 40 at predetermined separate locations to define electrodes 10. In either of such implementations, portions of substrate 40 may be removed, prior to or after electrodes 10 are provided, to define pockets (e.g. so as to increase the depth of spaces 60) with elevated regions 45 therebetween which correspond with the predetermined separate locations of electrodes 10.

As may be appreciated, in each of the above-noted approaches, electrodes 10 may be provided with substrate 40 advantageously oriented in a substantially planar layout. In turn, ease of manufacture as well as enhanced specification compliance and repeatability may be realized.

As shown in FIG. 4, each of the electrodes 10 may comprise opposing first surfaces 12 and second surfaces 14. As will be further described, upon removal of at least portions of substrate 40, the first surfaces 12 may be exposed to define the contact surfaces 12 of the neurostimulation electrode array 1 described above in relation to FIGS. 1 and 2.

With reference to FIGS. 3 and 4, electrical interconnection lines 30 may be interconnected to different ones of electrodes 10 and routed together along the first surface 42 of substrate 40. Such interconnections may be advantageously made with substrate 40 oriented in a substantially planar layout.

By way of example, electrical connection lines 30 may comprise insulated electrical wires having exposed ends that may be welded to different ones of the electrodes 10. Optionally, a strain relief member (not shown) may be provided over the interconnection regions (e.g. the welded regions). In one implementation, a silicone elastomer may be tacked over the interconnected regions to provide strain relief.

As shown in FIG. 5, after providing electrodes 10 on substrate 40 and interconnecting electrical connection lines 30 thereto, the substrate 40 may be configured to a predetermined configuration, wherein the first side 42 of the substrate 40 and exposed second surfaces 14 of the plurality of electrodes 10 face inward, and wherein the second side 44 of substrate 40 and the covered first surfaces 12 of the electrodes 10 face outward. By way of example, and as shown in FIG. 5, the substrate 40 may be rolled into a predetermined tubular configuration, wherein edges 46 and 48 of the substrate 40 are disposed in adjacent or overlapping relation to each other. In relation to the foregoing, substrate 40 and electrodes 10 may be provided to be sufficiently pliable for achieving the desired predetermined configuration.

As may be appreciated, the configured substrate 40 may define an internal volume 50 therein. Such volume 50 may correspond with all or at least a portion of the desired configuration of a carrier 20 of the neurostimulation electrode array 1 shown in FIGS. 1 and 2 above. In this regard, it should be appreciated that the spaces 60 provided between electrodes 10, including any above-noted pockets formed in substrate surfaces 42, may be filled with an electrically non-conductive material to define the insulator portions 22 of the carrier 20 of the neurostimulation electrode array 1. Relatedly, it should again be noted that the provision of elevated regions 45 may yield recessed positioning of contact surfaces 12 of electrodes 10 in a neurostimulation electrode array 1, as shown in FIGS. 1 and 2 above.

To form a carrier 20, a number of different approaches may be utilized. In one approach a carrier material may be introduced into the volume 50 after configuration of substrate 40. In another approach, a carrier material may be positioned over the first surface 42 of the substrate 40 and second surfaces 14 of electrodes 10 prior to the configuration of the substrate 40, wherein upon such configuration the carrier material is located within the internal volume 50. Combinations of such approaches, as well as other alternate approaches may also be employed in other embodiments.

In any case, upon providing a configured substrate 40 with carrier material disposed within a corresponding internal volume 50, the carrier material may be formed into a desired configuration, e.g. as defined by the inward-facing first surface 42 of substrate 40 and second surfaces 14 of electrodes 10. In conjunction with such formation, the carrier material may be physically interconnected to (e.g. bonded to) at least the exposed second surfaces 14 of electrodes 10. Further, the carrier material may form around the electrical connection lines 30, e.g. so as to sealably embed the electrical connection lines 30 within the carrier material.

The carrier material may be formed by applying a predetermined form of energy thereto so as to selectively modify a state of the carrier material. In turn, wherein after application of the energy the carrier material may maintain a desired shape, e.g. as defined by the internal volume 50, when substrate 40 is removed.

In the former regard, a number of different carrier formation approaches may utilized. In one approach, a configured substrate 40 (e.g. with electrodes 10 and electrical connection lines 30 interconnected thereto) may be positioned within a complimentary-shaped mold. Then, a carrier material may be injection-molded within the corresponding volume 50. Other approaches may include compression molding or casting of a catalyzed or two-part elastomer.

As noted above, after a carrier material is formed into a desired configuration of carrier 20, all or a desired portion of substrate 40 may be removed. In this regard, removal of the substrate 40 may be realized by exposing substrate 40 to an environmental condition selectively established to break-down and otherwise remove substrate 40 from the carrier 20 and electrodes 10, while not impacting the structural or operational integrity of carrier 20 and electrodes 10.

By way of example, in one implementation platinum-based electrodes 10 may be deposited on a copper-based or iron-based substrate 40, and a silicone-based carrier material may be utilized to form carrier 20. In turn, after formation of the carrier 20 a diluted nitric acid and/or hydrochloric acid may be employed to dissolve and thereby remove the substrate 40 from the carrier 20.

In another implementation, a catalyst, for example, a platinum compound, may be applied selectively to the spaces 60 between and after the formation of the electrodes 10. In turn, carrier 20 may be formed from a catalyzed elastomer. The use of a catalyst in spaces 60 promotes adhesion and polymerization of a catalyzed elastomer, reducing process time, improving mechanical integrity and enhancing insulation resistance between electrodes.

In further relation to the electrodes 10 shown in FIGS. 3-5, such electrodes 10 may be provided so that at least some of the corresponding first surfaces 12 have different areas. In this regard, the selective provision of different surface areas 12 advantageously facilitates the delivery of a desired electrical stimulation signal in relation to a given position along electrode 10. That is, the charge density and rate of charge transfer for a given electrode may be tailored to the desired intensity and rate of stimulation for the anatomical structure, for example the auditory nerve endings, in proximity to that electrode.

In conjunction with this feature, the provision of contact surfaces 12 having different areas may be achieved in number of ways. In particular, different contact surfaces 12 may be provided to have different corresponding areas per unit length as measured along a longitudinal axis AA and/or different corresponding lengths as measured along the length of longitudinal axis AA.

For example, in relation to the embodiment shown in FIG. 3, electrodes 10a and 10b may have corresponding contact surfaces 12a and 12b having different areas per unit length as measured along the longitudinal axis AA, as reflected by differences in their corresponding widths t₁ and t₂, respectively. As may be appreciated, differences in the widths of electrodes 10 may be reflected by corresponding differences in the radial distances of the contact surfaces 12 of electrodes 10 of the neurostimulation electrode array 1 shown in FIGS. 1 and 2. In another exemplary approach, electrodes 10a and 10b may have corresponding contact surfaces 12a and 12a having different areas due to differences in their corresponding lengths s₁ and s₂′ respectively.

The shape one or more of the contact surfaces 12 of electrodes 10 may also be selectively defined to be complex or non-uniform so as to take advantage of the physiology of stimulation. In this regard, the shape of contact surface 12 of a given electrode 18 may be defined to vary about or along a longitudinal axis AA. For example, the width of a given electrode 10 may increase and/or decrease as it progresses around a circumference of carrier 20, e.g. to yield a lens-like shape, a bowtie-like shape or other shapes. This shaping may be used to advantageously tailor the charge gradient to the relevant physiology, for example the local sensitivity of the auditory nerve. FIG. 3 illustrates in phantom lines how a given electrode 10c may be provided with a bow-tie configuration.

With further reference to FIGS. 3 and 5, substrate 40 may be shaped so that upon configuring the substrate into the configuration shown in FIG. 5, a frusto-conical internal volume 50 is defined therewithin. In turn, upon formation of the carrier 20 within the internal volume 50, and removal of substrate 40 therefrom, a neurostimulation electrode array 1 configuration may be realized that is generally tapered-down from the proximal end 2 to the distal end 3 thereof. For example, and as shown in the substantially planar layout of FIG. 3, the substrate 40 may be of an isosceles trapezoid configuration having an axis of symmetry BB.

The geometry of construction and methods of manufacture herein described allow greater flexibility in the design and application of electrodes for neurostimulation, making possible a wide range of applications offering stimulative therapies improved over previous devices.

1. An implantable neurostimulation electrode array, comprising:
a flexible, electrically non-conductive carrier; and,
a plurality of electrodes supportably interconnected to and spaced along a length of the carrier, wherein at least one of the following applies:
different ones of at least some of said plurality of electrodes comprise corresponding different contact surfaces having different configurations; and,
different pairs of at least some of said plurality of electrodes have corresponding spaces therebetween of different configurations.

2. An implantable neurostimulation electrode array as recited in claim 1, wherein different ones of at least some of said plurality of electrodes comprise corresponding different contact surfaces having different areas, and wherein said different contact surfaces have at least one of:
a different area per unit length of said carrier; and,
a different length as measures along a length of the carrier.

3. An implantable neurostimulation electrode array as recited in claim 1, wherein one or more of said plurality of electrodes comprises a corresponding contact surface having a non-uniform shape along or about a longitudinal axis the electrode array, and wherein said non-uniform shape has at least one of:
an increasing width as measured circumferentially; and,
a decreasing width as measured circumferentially.

4. An implantable neurostimulation electrode array as recited in claim 2, wherein said different contact surfaces comprise at least corresponding portions disposed at different distances from a longitudinal access of said carrier.

5. An implantable neurostimulation electrode array as recited in claim 2, wherein said each of different contact surfaces extend about and along a longitudinal access of said carrier.

6. An implantable neurostimulation electrode array as recited in claim 5, wherein each of said different contact surfaces are of an annular configuration.

7. An implantable neurostimulation electrode array as recited in claim 6, wherein each of said plurality of electrodes are of a ring-shaped configuration with said carrier being located therethrough.

8. An implantable neurostimulation electrode array as recited in claim 1, further comprising:
a plurality of electrical signal lines supportably interconnected to said carrier, wherein at least some of said electrical signal lines are electrically interconnected to different ones of said plurality of electrodes.

9. An implantable neurostimulation electrode array as recited in claim 8, wherein each of said plurality of electrical signal lines extend through at least a portion of said carrier.

10. An implantable neurostimulation electrode array as recited in claim 9, wherein each of said plurality of electrical signal lines is sealably disposed within said carrier.

11. An implantable neurostimulation electrode array as recited in claim 10, wherein said carrier is disposed between adjacent ones of said plurality of electrodes.

12. An implantable neurostimulation electrode array as recited in claim 1, wherein at least some of said plurality of electrodes comprise corresponding contact surfaces that are recessed relative to corresponding, electrically non-conductive portions of said array located therebetween.

13. An implantable neurostimulation electrode array as recited in claim 12, wherein said corresponding electrically non-conductive portions are integrally defined by said carrier.

14. An implantable neurostimulation electrode array as recited in claim 1, wherein said carrier has a maximum cross-dimension that is greater at a proximal end than at a distal end thereof.

15. An implantable neurostimulation electrode array as recited in claim 14, wherein said maximum cross-dimension of said carrier decreases along said length thereof from said proximal end to said distal end thereof.

16. An implantable neurostimulation electrode array as recited in claim 1, wherein said array is of a square-tooth configuration along a length thereof, with each of said plurality of electrodes being recessed relative to electrically non-conductive, insulator portions of said carrier located therebetween.

17. An implantable neurostimulation electrode array as recited in claim 16, wherein each of said plurality of electrodes is of a ring-shaped configuration.

18. An implantable neurostimulation electrode array as recited in claim 16, wherein said insulator portions are integrally defined by said carrier.

19. An implantable neurostimulation electrode array as recited in claim 1, wherein different pairs of at least some of said plurality of electrodes have corresponding spaces therebetween of different configurations, and wherein corresponding electrically non-conductive insulator portions are located in each of said spaces.

20. An implantable neurostimulation electrode array as recited in claim 1, wherein said carrier comprises a biocompatible material selected from a group consisting of:
a silicone based material;
a polyester urethane;
a polyether polyester copolymer;
a polypropylene oxide; and
wherein said plurality of electrodes each comprise a biocompatible metal selected from a group consisting of:
a platinum-based metal;
a platinum-iridium alloy;
an implantable stainless steel;
an implantable nickel-based alloy; and,
gold.

21.-44. (canceled)
